(19) United States
(12) Patent Application Publication
(10) Pub. No.: US 2004/0266677 A1
(43) Pub. Date: Dec. 30, 2004

(54) Method of diagnosing and treating lens illnesses using human HSF4 gene and coded product thereof

(76) Inventors: Xiangyin Kong, Shanghai (CN); Landian Hu, Shanghai (CN); Lei Bu, Shanghai (CN)

Correspondence Address: Min (Amy) S. Xu, DORSEY & WHITNEY LLP, Intellectual Property Department, 50 South Sixth street, Suite 1500, Minneapolis, MN, 55402-1498, US

(21) Appl. No.: 10/610,164

(22) Filed: Jun. 30, 2003

(51) Int. Cl. A61K 38/17; C12Q 1/68

(52) U.S. Cl. 514/12; 435/6

(57) ABSTRACT

The invention has disclosed a method for diagnosis of lens illnesses such as cataract. This method comprises the steps of detecting the HSF4 gene, transcript and/or protein in the subject and comparing it with the normal HSF4 gene, transcript and/or protein to determine whether there is any variation, wherein the variation indicates that the possibility of suffering lens illnesses, such as cataract, in the subject is higher than that in the normal population. The present invention also discloses the method and pharmaceutical composition for treating cataract and other lens illnesses.

### FIELD OF INVENTION

This invention relates to both biological engineering and medical fields. In particular, it relates to a method of diagnosing and treating lens illnesses, especially cataract, using human HSF4 gene and the coded product, and a pharmaceutical composition containing HSF4 gene and/or protein.

### TECHNICAL BACKGROUND

Cataract is a common eye disease, one of the leading causes of human blindness and one of the main diseases that severely influence the people's life. The main cause of cataract is the degeneration or sedimentation of crystallin in the lens. There are three kinds of crystallin: α-crystallin, β-crystallin, γ-crystallin. The γ-crystallin family consists of 7 members: A, B, C, D, E, F, S-crystallin. The previous reports show that the change of some crystallins may cause the lens illness and cataract. However, the mechanism of cataract is still unclear so far. Also the relationship between cataract and some special kind of crystallin is not reported.

Further, there is still no effective method to diagnose cataract early and to cure cataract by non-operative treatment in the art.

Therefore, there is an urgent need to develop new and efficient methods to diagnose and cure cataract, the relative pharmaceuticals, and diagnostic technology and reagents.

### SUMMARY OF INVENTION

One purpose of the invention is to provide a new diagnostic method, especially for early diagnosis, and detection kit for cataract and other lens illnesses.

Another purpose is to provide a new method to treat cataract and other lens illnesses.

Still another purpose is to provide a pharmaceutical composition to treat cataract and other lens illnesses.

In the first aspect, the invention provides a method for determining the cataract susceptibility in a subject comprising the steps of:

detecting the HSF4 gene, transcript and/or protein in said subject and comparing it with the normal HSF4 gene, transcript and/or protein to determine whether there is any difference,

wherein said difference indicates that the possibility of suffering cataract in said subject is higher than that in the normal population.

In a preferred embodiment, the HSF4 gene or transcripts, including HSF4a, HSF4b and other different transcripts, are detected, and compared with the normal HSF4 nucleotide sequence to determine the difference. More preferably, said difference is selected from the group consisting of: in position 348 of SEQ ID NO: 1, T→C; in position 115 of SEQ ID NO: 2, Leu→Pro.

In the second aspect, the invention provides a method for treating lens illnesses comprising the step of administrating a safe and effective amount of normal HSF4 protein to the patient in need of said treatment. Preferably, the HSF4 protein is administrated topically to the eyes.

In the third aspect, the invention provides a pharmaceutical composition comprising a safe and effective amount of HSF4 protein and a pharmaceutically acceptable carrier. Preferably, said pharmaceutical composition is eyedrops or eye ointments.

In the fourth aspect, the invention provides a kit for detecting lens illnesses comprising the primers that specifically amplify the HSF4 gene or transcript. Preferably, the kit further comprises a probe that binds to the site of mutation and/or an enzyme recognizing and cutting the site of mutation. More preferably, the enzyme is BsrS I and the primers are SEQ ID NOs: 7 and 8.

In view of the technical teaching of the invention, the other aspects of the invention will be apparent to the skilled in the art.

### DESCRIPTION OF DRAWINGS

FIG. 1 shows the pedigree of an autosomal dominant cataract family.

FIG. 2 shows the pathological changes in the eyes of cataract patients.

FIG. 3 shows the genetic linkage analysis results.

FIG. 4 shows the sequence changes of the HSF4 gene. The sequence in the normal is “. . . CTACTG . . . ”, while in patients from the cataract family, the sequence in one of the two chromosomes changes into “. . . CTACCG . . . ”. This mutation causes Leu→Pro change in the encoded product.

### DETAILED DESCRIPTION OF INVENTION

HSF4 is an identified protein. The following is its basic information.

Name: Homo sapiens for transcription factor HSF4 (Heat Shock transcription Factor 4)

NCBI: Contig: NT_{—}010478

mRNA: Homo sapiens heat shock transcription factor 4 (HSF4), mRNA gil45576501|reflNM_{—}001538.1|[4557650]

The mRNA sequence of HSF4 is showed in SEQ ID NO: 1. The ORF is position 5-1393, coding a full-length protein having 463 amino acids (SEQ ID NO: 2). Other information about HSF4 is available from http://www.ncbi.nlm.nih.gov.

The inventors have found and proved that HSF4 has a close relationship with cataract for the first time. In addition, its new function is found: The changes of HSF4 cause the lens illness and cataract directly. On the basis of this discovery, the inventors finished this invention.

By linkage analysis, candidate gene screen and sequencing, we proved that the mutation in HSF4 leads to the phenotypes of cataract and pathological changes in lens. Our study shows that human HSF4 not only has a close relationship with heat shock protein, but also can control the stability of crystallin. The stability of HSF4 plays a key role in keep the normal physiological behavior of lens.

By comparing the protein homology, we have found that human HSF4 is very conservative. So the mutation of HSF4 is one of the direct reasons leading to cataract and other lens illnesses in human. One can develop new drugs and means according to HSF4 gene and its expression product for the diagnosis and treatment of the lens illnesses in human.

The full-length human HSF4 nucleotide sequence or its fragment of the invention can be prepared by PCR amplification, recombinant method and synthetic method. For PCR amplification, one can obtain said sequences by designing primers based on the nucleotide sequence disclosed in the invention, especially the sequence of ORF, and using cDNA library commercially available or prepared by routine techniques known in the art as a template. When the sequence is long, it is usually necessary to perform two or more PCR amplifications and link the amplified fragments together in the correct order.

Once the sequence is obtained, a great amount of the sequences can be produced by recombinant methods. Usually, said sequence is cloned in a vector which is then transformed into a host cell. Then the sequence is isolated from the amplified host cells using conventional techniques.

Further, the sequence can be produced by synthesis. Typically, several small fragments are synthesized and linked together to obtain a long sequence.

The HSF4 encoding sequence can be inserted into an appropriate expression vector and transferred into a host cell. Then the HSF4 protein can be isolated from the culture.

Based on the new discovery of the invention, the HSF4 protein or polypeptide have various uses including but not limited to: curing disorders such as cataract caused by low or no activity of HSF4 protein (using directly as a medicine), and screening out antibodies, polypeptides or ligands which promote the function of HSF4. The expressed recombinant HSF4 protein can be used to screen polypeptide library to find therapeutically valuable polypeptide molecules which activate the function of HSF4 protein.

In another aspect, the invention also includes polyclonal and monoclonal antibodies, preferably monoclonal antibodies, which are specific for polypeptides encoded by human HSF4 DNA or fragments thereof. By “specificity”, it is meant an antibody which binds to the human HSF4 gene products or a fragments thereof. Preferably, the antibody binds to the human HSF4 gene products or fragments thereof and does not substantially recognize nor bind to other antigenically unrelated molecules. Antibodies which bind to human HSF4 and block human HSF4 protein and those which do not affect the human HSF4 function are included in the invention.

The present invention includes not only intact monoclonal or polyclonal antibodies, but also immunologically-active antibody fragments, e.g., a Fab′ or (Fab)₂ fragment, an antibody heavy chain, an antibody light chain, a genetically engineered single chain Fv molecule, or a chimeric antibody.

The antibodies in the present invention can be prepared by various techniques known to those skilled in the art. For example, purified human HSF4 gene products, or its antigenic fragments can be administrated to animals to induce the production of polyclonal antibodies. Similarly, cells expressing human HSF4 or its antigenic fragments can be used to immunize animals to produce antibodies. Various adjuvants, e.g., Freund's adjuvant, can be used to enhance the immunization.

The antibodies of the invention can be monoclonal antibodies which can be prepared by using hybridoma technique. Antibodies of the invention comprise those which block human HSF4 function and those which do not affect human HSF4 function. Antibodies in the invention can be produced by routine immunology techniques and using fragments or functional regions of human HSF4 gene product. These fragments and functional regions can be prepared by recombinant methods or synthesized by a polypeptide synthesizer. The antibodies binding to unmodified human HSF4 gene product can be produced by immunizing animals with gene products produced by prokaryotic cells (e.g., E. coli), and the antibodies binding to post-translationally modified forms thereof can be acquired by immunizing animals with gene products produced by eukaryotic cells (e.g., yeast or insect cells).

The antibody against human HSF4 protein can be used in immunohistochemical method to detect the presence of HSF4 protein in the biopsy specimen. The preferred anti-HSF4 antibody does not recognize the normal HSF4 but recognize the mutated HSF4, e.g., the one having Leu115→Pro 115 mutation in SEQ ID NO: 2. Alternatively, The preferred anti-HSF4 antibody recognizes the normal HSF4 but does not recognize the mutated HSF4. Using the recognition difference between the normal and mutated HSF4, one can easily detect the susceptibility of cataract on the level of protein.

The substances which act with HSF4 protein, e.g., inhibitors, agonists and antagonists, can be screened out by various conventional techniques, using the protein of the invention.

The HSF4 protein, antibody, inhibitor, agonist or antagonist of the invention provide different effects when administrated in therapy. Usually, these substances are formulated with a non-toxic, inert and pharmaceutically acceptable aqueous carrier. The pH typically ranges from 5 to 8, preferably from about 6 to 8, although pH may alter according to the property of the formulated substances and the diseases to be treated. The formulated pharmaceutical composition is administrated in conventional routine including, but not limited to, intramuscular, intravenous, subcutaneous, or topical administration including circumocular, retrobulbar and intraocular injection. The topical administration at eyes is preferred.

The normal HSF4 can be directly used for curing disorders, e.g., lens illnesses including cataract. The HSF4 protein of the invention can be administrated in combination with other cataract medicaments.

The invention also provides a pharmaceutical composition comprising safe and effective amount of HSF4 protein in combination with a suitable pharmaceutical carrier. Such a carrier includes but is not limited to saline, buffer solution, glucose, water, glycerin, ethanol, or the combination thereof. The pharmaceutical formulation should be suitable for the delivery method. The pharmaceutical composition of the invention may be in the form of injections which are made by conventional methods, using physiological saline or other aqueous solution containing glucose or auxiliary substances. The pharmaceutical compositions in the form of eyedrops, eye ointments, tablet or capsule may be prepared by routine methods. The pharmaceutical compositions, e.g., eyedrops, eye ointments, injections, solutions, tablets, and capsules, should be manufactured under sterile conditions. The active ingredient is administrated in therapeutically effective amount, e.g., from about 0.1 ug to 10 mg per kg body weight per day. Moreover, the polypeptide of the invention can be administrated together with other therapeutic agents.

When using pharmaceutical composition, the safe and effective amount of the HSF4 protein or its antagonist or agonist is administrated to mammals. Typically, the safe and effective amount is at least about 0.1 ug/kg body weight and less than about 10 mg/kg body weight in most cases, and preferably about 0.1-100 ug/kg body weight. Of course, the precise amount will depend upon various factors, such as delivery methods, the subject health, and the like, and is within the judgment of the skilled clinician.

The human HSF4 polynucleotides also have many therapeutic applications. Gene therapy technology can be used in the therapy of abnormal cell proliferation, development or metabolism, which is caused by the loss of HSF4 expression or the expression of abnormal or non-active HSF4. The methods for constructing a recombinant virus vector harboring HSF4 gene are described in the literature (Sambrook, et al.). In addition, the recombinant HSF4 gene can be packed into liposome and then transferred into the cells.

The methods for introducing the polynucleotides into tissues or cells include: directly injecting the polynucleotides into tissue in the body, in vitro introducing the polynucleotides into cells with vectors, such as virus, phage, or plasmid, and then transplanting the cells into the body.

The invention further provides diagnostic assays for quantitative and in situ measurement of HSF4 protein level. These assays are well known in the art and include FISH assay and radioimmunoassay.

A method of detecting the presence of HSF4 protein in a sample by utilizing the antibody specifically against HSF4 protein comprises the steps of: contacting the sample with the antibody specifically against HSF4 protein; observing the formation of antibody complex which indicates the presence of HSF4 protein in a sample.

The polynucleotide encoding HSF4 protein can be used in the diagnosis and treatment of HSF4 protein related diseases. In respect of diagnosis, the polynucleotide encoding HSF4 can be used to detect whether HSF4 is expressed or not, and whether the expression of HSF4 is normal or abnormal, e.g., in the case of diseases. HSF4 DNA sequences can be used in the hybridization with biopsy samples to determine the expression of HSF4. The hybridization methods include Southern blotting, Northern blotting and in situ blotting, etc., which are public and sophisticated techniques. The corresponding kits are commercially available. A part of or all of the polynucleotides of the invention can be used as probe and fixed on a microarray or DNA chip for analyzing the differential expression of genes in tissues and for the diagnosis of genes. The HSF4 specific primers can be used in RNA-polymerase chain reaction and in vitro amplification to detect the transcripts of HSF4.

The invention also provides a method for detecting the SNP in human HSF4 gene, comprising the steps of: (a) determining the nucleotide on the position 348 of SEQ ID NO: 1 of human HSF4; and (b) determining whether said position has a SNP. One SNP is T348→C348.

Further, detection of the mutation of HSF4 gene is useful for the diagnosis of cataract. The detection may focus on cDNA or genomic DNA. Some of primers used to amplify the genomic DNA are listed in SEQ ID NO: 3 and 8. The mutation forms of HSF4 include site mutation, translocation, deletion, rearrangement and any other mutations compared with the normal wild-type HSF4 DNA sequence. The conventional methods, such as Southern blotting, DNA sequencing, PCR and in situ blotting, can be used to detect mutation. Moreover, mutation sometimes affects the expression of protein. Therefore, Northern blotting and Western blotting can be used to indirectly determine whether the gene is mutated or not.

The invention is further illustrated by the following examples. It is appreciated that these examples are only intended to illustrate the invention, but not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### EXAMPLE 1:

The identification of an autosomal dominant cataract family

1.1 subject

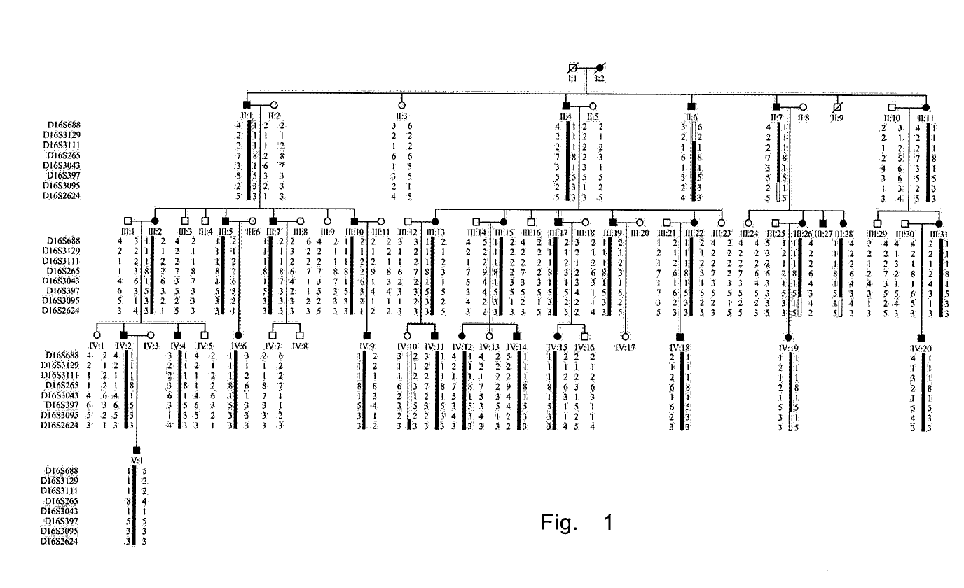

We identified a large five-generation Chinese family affected with congenital cataract, total 65 people in this family. Among them, 31 were affected with lamellar cataract (FIG. 1).

1.2 Clinical examination

1.2.1 Sight

Examining the naked vision and corrected visual acuity with visual acuity chart.

1.2.2 Dilated pupil examination

Examining lens opacity condition with slit lamp or pocket lamp and observing fundus condition by using direct funduscope.

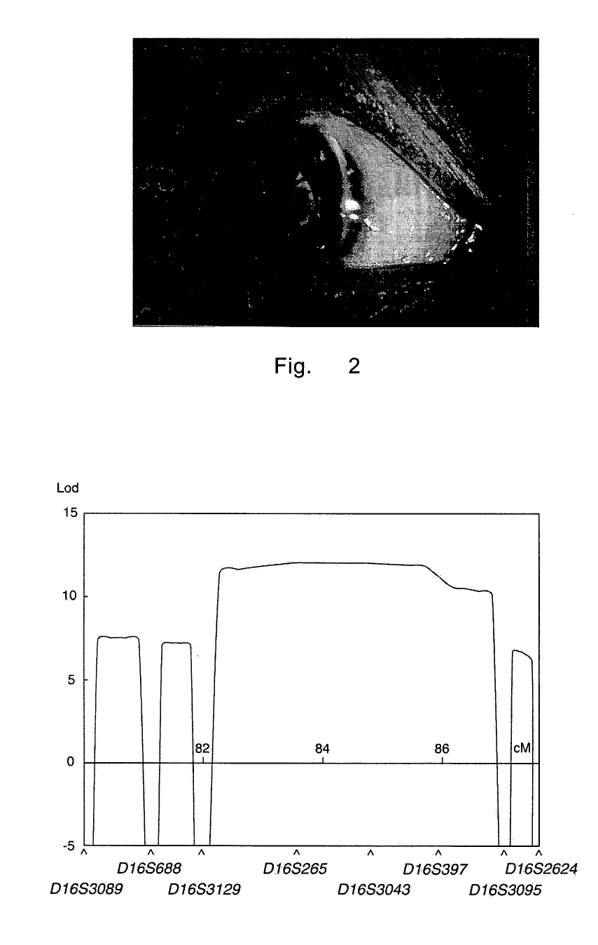

In this large Chinese family, the lens opacity was characterized by perinuclear a change, that mean a turbid fetal nucleus surrounded a transparent embryonic nucleus and outside was transparent crystal structure (FIG. 2). Among all the patients investigated, lens opacity has a coincident form and only has a little difference in level, except a ten-month old patient showed a white spot-shaped opacity.

The disease characteristic in this family was that both two eyes were affected with slow progress and a middling effect on vision; the naked vision was usually between 0.1 and 0.3; often accompanied with low myopia. Restricted by medicine and traffic condition in that area, they usually went to see a doctor when they went to school after 7 years old and found their vision was so poor that they couldn't study normally as others.

Among all the patients, there was no other congenital abnormity in eye part except a 6 years old girl who was affected with congenital blepharoptosis in one eye. Other whole-body illnesses were not found in all the patients.

1.3 segregation analysis

To do segregation analysis with Li-Mantal-Gart and SEGRAN B methods separately, analyzing genetic type of this family.

1.3.1 Li-Mantal-Gart method

For this family had been identified, it was analyzed by Singles method, also called Li-Mantal Gart method. The results were showed in the following:

| | | | | The family number only |
|---|---|---|---|---|
| Sib number in | Family | Total sib | Patient | concluding one patient |
| each family | number | number | number | in their sibs |
| 1 | 6 | 6 | 6 | 6 |
| 2 | 4 | 8 | 4 | 4 |
| 3 | 1 | 3 | 2 | 0 |
| 4 | 2 | 8 | 5 | 0 |
| 7 | 3 | 21 | 15 | 0 |
| Total | 16 | 46 (J) | 31 (R) | 10 (J) |

Segregation ratio P = (R − J)/(T − J)
P: Estimated segregation ration needed corrected
R: The total number of affected children
T: The total number of siblings
J: The family number only concluding one patient in their sibling

Variance SP²=(R-I)(T-R)/(T-J)³

Standard error SE=\sqrt{SP²}

95% confidence interval P±1.96SE

95% confidence interval 45.185%-77.0152%

The estimated P included 0.25, so we supposed that it was an autosomal dominant genetic disease.

After analyzed by SEGRAN B method, it was confirmed that it was an autosomal dominant genetic disease

1.4 pathologic examinations

To deal two patients in this family with ECCE+IOL insertion, take the lens out, fix with eyeball fixing liquid, stain with eosin and then observe under light microscope. To with the control lens, we used the same method to do pathologic observation as contrast.

The result showed that there were some blue spotted basophilic particles in the nuclear region of the patients.

### EXAMPLE 2:

HSF4 mutation is identified as the direct cause for cataract.

2.1 Genetic linkage analysis

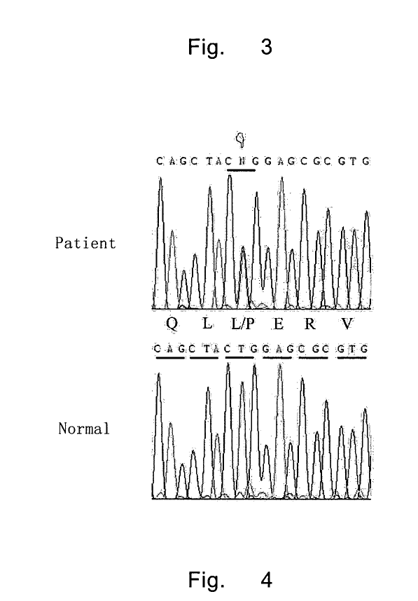

Microsatellite markers were used in the genetic linkage analysis of this family. Totally, 384 microsatillite primers were used to screen the whole genome, we localized the cataract locus onto chromosome 16. By designing more primers, the cataract locus was mapped to the 5.11-cM interval between D16S3129 and D16S3095 (FIGS. 1 and 3). In this region, there are about 7 million base pairs and more than 130 genes.

2.2 Candidate gene

We designed PCR primers to screen candidate genes firstly, then sequenced the PCR products. After sequencing with 9 pairs of primers for HSF4, we found that HSF4 had a close relationship with cataract.

Sequencing of PCR products generated by two pairs of primers, the products were 483 and 556 bp separately, showed that a T to C transition at nucleotide 348 (SEQ ID NO: 1) of HSF4 mRNA (NM_{—}001538) in all the affected. This mutation is predicted to result in a Leu115Pro substitution (FIG. 4)

| | | | |
|---|---|---|---|
| Name | Sequence(5′→3′) | Bp | Number |

| | | | | |
|---|---|---|---|---|
| Pair 1 | HSF4_E×23_Fwd | agcgcaggactggccgtgag | 20 | SEQ ID NO: 3 |
| | HSF4_E×23_Rev | gggactgggtcgcaggagca | 20 | SEQ ID NO: 4 |
| Pair 2 | HSF4_E×34_Fwd | agtgctgccccagtatttcaag | 22 | SEQ ID NO: 5 |
| | HSF4_E×34_Rev | gccagttatggtctcatcccg | 21 | SEQ ID NO: 6 |

Structure analysis showed that the protein structure in HSF4 region was changed. Comparing with other species, we found the amino acid was conserved in the mutation region and within the key DNA-binding domains of HSF4.

In addition, this change was not found in all 200 normal controls selected randomly. This result suggests that HSF4 has a close relationship with human cataract. The encoding product of this gene plays an important role on human cataract.

### EXAMPLE 3:

Cataract detection Kit:

As FIG. 4 showed: The sequence in normal people is “. . . CTACTG . . . ”, while, in cataract family patients, the sequence in one of the two chromosomes changes into “. . . CTACCG . . . ”. This mutation is predicted to result in a Leu115Pro substitution, leading to a cataract phenotype. So we designed primers (such as SEQ ID NO: 3, 4, 5 and 6) according to this mutation, and then amplified the DNA samples from patients, finally examined the PCR products,

Additionally, the sequence “ACTGG” in normal person can be identified by restriction endonuclease “BsrS I”. When the T is substituted by C in 348 site, the enzyme cleavage site changes as a result.

A kit which can be used for 100 samples was prepared which contained the components as shown in the following table:

| Name | Sequence (5′→3′) | Number | Quantity |
|---|---|---|---|
| Primer F | 5′-agtgctgccccagtatttcaag-3′ | SEQ ID NO: 7 | 100 pmol |
| Primer R | 5′-gggactgggtcgcaggagca-3′ | SEQ ID NO: 8 | 100 pmol |
| BsrS I | | | 10 U |
| PCR buffer | | | 5 ml |
| Enzyme buffer | | | 5 ml |

When we amplified HSF4 with this kit to examine cataract, using blood DNA from patients as samples, the products were 356 bp. After cutting products from the normal with BsrS I, the amplification products degraded into four fragments: 252 bp, 87 bp, 10 bp and 7 bp (The cleavage sites were positions 10, 262 and 349).

The enzyme cleavage site could not be identified by BsrS I in patients, for the T was substituted by C in their chromosomes. Thus, the PCR products of 356 bp degraded into three fragments: 339 bp, 10 bp and 7 bp (The cleavage sites were positions 10 and 349). They were easily detectable by electrophoresis.

### EXAMPLE 4:

The preparation of pharmaceutical composition

The HSF4 protein was obtained by constructing expression carrier containing human HSF4 gene and expressing the protein, or by separating it from human and animal nature proteins with liquid chromatography. The purified HSF4 was made into injection and injected to muscle under the patient eyes. This method supplied the normal HSF4 protein to patients and their cataract was ameliorated or even cured.

All the documents cited herein are incorporated into the invention as reference, as if each of them is individually incorporated. Further, it would be appreciated that, in the above teaching of the invention, the skilled in the art could make certain changes or modifications to the invention, and these equivalents would still be within the scope of the invention defined by the appended claims of the present application.

What is claimed is:

1. A method for determining the cataract susceptibility in a subject comprising the steps of:
detecting the HSF4 gene, transcript and/or protein in said subject and comparing it with the normal HSF4 gene, transcript and/or protein to determine whether there is any difference,
wherein said difference indicates that the possibility of suffering cataract in said subject is higher than that in the normal population.

2. The method of claim 1 wherein the HSF4 gene or transcript is detected, and compared with the normal HSF4 nucleotide sequence to determine the difference.

3. The method of claim 1 wherein said difference is selected from the group consisting of:
in position 348 of SEQ ID NO: 1, T→C;
in position 115 of SEQ ID NO: 2, Leu→Pro.

4. A method for treating lens illnesses comprising step of administrating a safe and effective amount of normal HSF4 protein to the patient in need of said treatment.

5. The method of claim 4 wherein the HSF4 protein is administrated topically to the eyes.

6. A pharmaceutical composition comprising a safe and effective amount of HSF4 protein and a pharmaceutically acceptable carrier.

7. The pharmaceutical composition of claim 6 which is selected from the group consisting of eyedrops and eye ointments.

8. A kit for detecting lens illnesses comprising the primers which specifically amplify the HSF4 gene or transcript.

9. The kit of claim 8 which further comprises a reagent selected from the group consisting of:
(a) a probe that binds to the site of mutation; and
(b) a restriction enzyme recognizing and cutting the site of mutation.

10. The kit of claim 9, wherein the mutation is T→C in position 348 of SEQ ID NO: 1.

11. The kit of claim 9 wherein the enzyme is BsrS I.

12. The kit of claim 8 wherein the primers are SEQ ID NOs: 7 and 8.


SEQUENCE LISTING (8 sequences)

SEQ ID NO: 1 - DNA, 1555 bp, Homo sapiens
  CDS (5)..(1393)
ccggatggtgcaggaagcgccagctgcgctgcccacggagccaggccccagccccgtgcctgccttcctcggcaagctatgggcgctggtgggggacccaggcacagaccacctgatccgctggagcccgagcgggaccagtttcctcgtaagcgaccagagccgtttcgccaaggaagtgctgccccagtatttcaagcatagcaacatggcgagcttcgtgcgccaactcaacatgtacggttttcggaaggtggtgagcatcgagcagggcggcctgcttaggccggagcgcgaccacgtcgagttccagcacccgagcttcgtgcgcggccgcgagcagctactggagcgcgtgcggcgcaaggtgcccgcgctgcgcggcgacgacggccgctggcgcccggaggacctgggtcgactactgggcgaggtgcaggctttgcggggagtgcaggagagcaccgaggcgcggctgcgggagctcaggcagcagaacgagatcttgtggcgggaggtggtgacacttcggcagagccacggtcagcagcaccgggtcattggcaagctgatccagtgtctctttgggccacttcaggcggggccgagcaatgcaggaggcaagagaaagctgtccctgatgctggatgaggggagctcatgcccaacacctgccaagttcaacacctgccctctacctggtgcccttctgcaggacccctacttcatccagtcgccttctacttacagcctctcccagagacaaatttgggccttagccctcacagggccaggggccccatcatctctgacatcccagaagactctccatcccctgaggggaccaggctttctccctccagtgatggcaggagcccccccgccactgcctgtggctgtggtgcaggccatcctggaagggaaagggagcttcagccccgaggggcccaggaatgcccaacagcctgaaccaggggatcccagggagatacctgacagggggcctctgggcctggaaagcggggacaggagcccagagagtctgctgcctccgatgctgcttcagccccctcaagaaagtgtggaacctgcagggcctctagatgtgctgggccccagtctccaagggcgagaatggaccctgatggacttggacatggagctgtccttgatgcagcccttggttccagagcggggtgagcctgagctggcggtcaaggggttaaattctccaagcccagggaaggaccccacgctcggggccccactcctgctggatgtccaggcggccttgggaggcccagccctgggcctgcctggggctttaaccatttatagcactcctgagagccggactgcctcctacttgggcccggaagccagtccctccccctaagaccccgcgcctctgaaggggcttggaaccagtccgccgctgcacatccttcttggcttcctggccgcctacgggggtgagcgaagcccccactactaaatggcctctctccactaccccgactatccctgcacataaactccgtttttttttttcacc

SEQ ID NO: 2 - protein, 463 aa, Homo sapiens
MVQEAPAALPTEPGPSPVPAFLGKLWALVGDPGTDHLIRWSPSGTSFLVSDQSRFAKEVLPQYFKHSNMASFVRQLNMYGFRKVVSIEQGGLLRPERDHVEFQHPSFVRGREQLLERVRRKVPALRGDDGRWRPEDLGRLLGEVQALRGVQESTEARLRELRQQNEILWREVVTLRQSHGQQHRVIGKLIQCLFGPLQAGPSNAGGKRKLSLMLDEGSSCPTPAKFNTCPLPGALLQDPYFIQSPSTYSLSQRQIWALALTGPGAPSSLTSQKTLHPLRGPGFLPPVMAGAPPPLPVAVVQAILEGKGSFSPEGPRNAQQPEPGDPREIPDRGPLGLESGDRSPESLLPPMLLQPPQESVEPAGPLDVLGPSLQGREWTLMDLDMELSLMQPLVPERGEPELAVKGLNSPSPGKDPTLGAPLLLDVQAALGGPALGLPGALTIYSTPESRTASYLGPEASPSP

SEQ ID NO: 3 - DNA, 20 bp, Artificial Sequence
  misc_feature (1)..(20): primer
agcgcaggactggccgtgag

SEQ ID NO: 4 - DNA, 20 bp, Artificial Sequence
  misc_feature (1)..(20): primer
gggactgggtcgcaggagca

SEQ ID NO: 5 - DNA, 22 bp, Artificial Sequence
  misc_feature (1)..(22): primer
agtgctgccccagtatttcaag

SEQ ID NO: 6 - DNA, 21 bp, Artificial Sequence
  misc_feature (1)..(21): OyI<
gccagttatggtctcatcccg

SEQ ID NO: 7 - DNA, 22 bp, Artificial Sequence
  misc_feature (1)..(22): primer
agtgctgccccagtatttcaag

SEQ ID NO: 8 - DNA, 20 bp, Artificial Sequence
  misc_feature (1)..(20): primer
gggactgggtcgcaggagca